SEQUENCE LISTING (4 sequences)

SEQ ID NO: 1 - DNA, 20 bp, Artificial sequence
  CP1  Primer designed to hybridize to the Chlamydia pneumoniae major outer membrane protein sequence
ttacaagccttgcctgtagg

SEQ ID NO: 2 - DNA, 21 bp, Artificial sequence
  CP2  Primer designed to hybridize to the Chlamydia pneumoniae major outer membrane protein sequence
gcgatcccaaatgtttaaggc

SEQ ID NO: 3 - DNA, 21 bp, Artificial sequence
  CPC  Primer designed to hybridize to the Chlamydia pneumoniae major outer membrane protein sequence
ttattaattgatggtacaata

SEQ ID NO: 4 - DNA, 21 bp, Artificial sequence
  CPD  Primer designed to hybridize to the Chlamydia pneumoniae major outer membrane protein sequence
atctacggcagtagtatagtt

(19) United States
(12) Patent Application Publication
(10) Pub. No.: US 2006/0141470 A1
(43) Pub. Date: Jun. 29, 2006

(54) Chlamydia pneumoniae associated chronic intraocular disorders and treatment thereof

(76) Inventor: Murat V. Kalayoglu, Boston, MA (US)

Correspondence Address: GOODWIN PROCTER LLP;PATENT ADMINISTRATOR, EXCHANGE PLACE, BOSTON, MA, 02109-2881, US

(21) Appl. No.: 10/545,359

(22) PCT Filed: Feb. 12, 2004
(86) PCT No.: PCT/US04/04108

§ 371 Date: Aug. 11, 2005

Related U.S. Application Data
(60) Provisional application No. 60/447,367, filed on Feb. 14, 2003.
(60) Provisional application No. 60/503,402, filed on Sep. 16, 2003.

(51) Int. Cl. C12Q 1/68; G01N 33/554; G01N 33/569

(52) U.S. Cl. 435/6; 435/7.32

(57) ABSTRACT

Infection by Chlamydia pneumoniae has been identified as a risk factor associated with the pathogenesis of certain chronic intraocular disorders, for example, age-related macular degeneration. The invention provides methods of identifying individuals at risk of developing chronic intraocular disorders, and methods of delaying and/or preventing the onset of such chronic intraocular disorders.

### REFERENCE TO RELATED APPLICATIONS

This application claims priority to U.S. Provisional Application Ser. No. 60/447,367, filed Feb. 14, 2003 and U.S. Provisional Application Ser. No. 60/503,402, filed Sep. 16, 2003, the disclosures of which are incorporated by reference herein.

### FIELD OF THE INVENTION

The present invention relates generally to the field of chronic intraocular disorders. More particularly, the invention relates to methods for identifying individuals at risk of developing a chronic intraocular disorder associated with Chlamydia pneumoniae, and to methods of preventing, slowing or stopping the development of such a disorder.

### BACKGROUND

There are a variety of chronic intraocular disorders, which, if untreated, may lead to partial or even complete vision loss. One prominent chronic intraocular disorder is age-related macular degeneration, which is the leading cause of blindness amongst elderly Americans affecting a third of patients aged 75 years and older (Fine et al. (2000) NEW ENGL. J. MED. 342: 483-492). There are two forms of age-related macular degeneration, a wet form, which is associated with the formation of neovasculature in the choroid (also known as the neovascular form of age-related macular degeneration), and a dry form, which is not associated with the formation of choroidal neovasculaturization. The wet form accounts for approximately 90% of the severe vision loss associated with age-related macular degeneration.

Multiple risk factors have been identified in the pathogenesis of age-related macular degeneration. In addition to age, another established risk factor is a history of tobacco use. Case-control and population-based studies strongly support an association between age-related macular degeneration and smoking, with odds ratios greater than 2.0 for developing age-related macular degeneration in smokers compared to non-smokers (Seddon et al. (1996) J. AM. MED. ASSOC. 276: 1141-1146; Christen et al. (1996)) J. AM. MED. ASSOC. 276: 1147-1151). Other associated risk factors include hypertension, atherogenic lipid profile and a history of atherosclerotic vascular disease (Hawkins et al. (1999) MOL. VIS. 5: 26).

Currently, treatment of the dry form of age-related macular degeneration includes administration of antioxidant vitamins and/or zinc. Treatment of the wet form of age-related macular degeneration, however, has proved to be more difficult. Currently, two separate methods have been approved in the United States of America for treating the wet form of age-related macular degeneration. These include laser photocoagulation and photodynamic therapy using a benzoporphyrin derivative photosensitizer. During laser photocoagulation, thermal laser light is used to heat and photocoagulate the neovasculature of the choroid. A problem associated with this approach is that the laser light must pass through the photoreceptor cells of the retina in order to photocoagulate the blood vessels in the underlying choroid. As a result, this treatment destroys the photoreceptor cells of the retina creating blind spots with associated vision loss. During photodynamic therapy, a benzoporphyrin derivative photosensitizer is administered to the individual to be treated. Once the photosensitizer accumulates in the choroidal neovasculature, non-thermal light from a laser is applied to the region to be treated, which activates the photosensitizer in that region. The activated photosensitizer generates free radicals that damage the vasculature in the vicinity of the photosensitizer (see, U.S. Pat. Nos. 5,798,349 and 6,225,303). This approach is more selective than laser photocoagulation and is less likely to result in blind spots. Under certain circumstances, this treatment has been found to restore vision in patients afflicted with the disorder (see, U.S. Pat. Nos. 5,756,541 and 5,910,510).

However, there is still an ongoing need for methods of identifying individuals at risk of developing as well as methods of preventing the onset of chronic ocular disorders, and once diagnosed, the treatment of such a disorder.

### SUMMARY OF THE INVENTION

The present invention is based, in part, upon the discovery that Chlamydia pneumoniae infection is a risk factor associated with the development of certain chronic intraocular disorders. As a result, the invention provides a method of determining whether an individual is at risk of developing a chronic intraocular disorder. In addition, because Chlamydia pneumoniae is a treatable risk factor, the invention provides a method of preventing the onset and/or development of certain chronic intraocular disorders.

In one aspect, the invention provides a method of determining whether a mammal, for example, a primate, more specifically, a human, is at risk of developing a chronic intraocular disorder. Exemplary chronic intraocular disorders include without limitation, age-related macular degeneration, uveitis syndromes (for example, chronic iridocyclitis or chronic endophthalmitis), choroidopathies (for example, “White-dot” syndromes including, but not limited to, acute multifocal posterior placoid), ocular glaucomas (for example, inflammatory glaucomas), retinopathies (for example, cystoid macular edema, central serous choroidopathy and presumed ocular histoplasmosis syndrome), retinal vascular disease (for example, diabetic retinopathy, Coat's disease and retinal arterial macroaneurysm), and cataracts. The method comprises detecting in a sample harvested from the mammal the presence of Chlamydia pneumoniae or a Chlamydia pneumoniae specific agent. The presence and/or concentration of Chlamydia pneumoniae or a Chlamydia pneumoniae specific agent in the sample is indicative that the mammal is at risk of developing the chronic intraocular disorder.

The sample tested may be a tissue or body fluid sample. In particular, the method of the invention detects the presence of Chlamydia pneumoniae or a Chlamydia pneumoniae specific agent in a body fluid sample including, for example, blood, serum, plasma, urine, lacrimal fluid, vitreous, aqueous, and synovial fluid. In addition, the method of the invention detects the presence of Chlamydia pneumoniae or a Chlamydia pneumoniae specific agent in a tissue sample including, for example, skin, conjunctiva, cornea, sclera, uvea, retina, choroid, neovascular tissue, and optic nerve.

Chlamydia pneumoniae is a relatively recent addition to the Chlamydia genus. Although first recognized as causing community acquired pneumonia in the 1980s, this pathogen has quickly become associated with other respiratory tract diseases such as sinusitis, pharyngitis and bronchitis (Kalayoglu (2002) CURRENT DRUG TARGETS—Inflammation & Allergy 1: 249-255; Grayston (1992) CLIN. INFECT. DIS. 15: 757-761). More recently, the pathogen has been found to be associated with the development of atherosclerosis (see, Kalayoglu et al. (2002) J. AM. MED. ASSOC. 288: 2724-2731, Byrne & Kalayoglu (1999) AM. HEART J. 138: S488-S490; Byrne et al. (2000) J. INFECT. DIS. 181: S490-491), and certain ocular disorders, for example, acute anterior uveitis (see, Huhtinen et al. (2001) INVEST. OPHTH. VIS. SCI. 42: 1816-1819), chronic conjunctivitis (see, Lietman et al. (1998) CLIN. INFECT. DIS. 26(6): 1335-1340), non-arteritic anterior ischemic optic neuropathy (see, Weger et al. (2002) OPHTHALMOL. 109: 749-752), and branch retinal vein occlusion (see, Jumper et al. (2002) Symposium 5: Retinal Vascular Disease: Branch Retinal Vein Occlusion is Associated with Serologic Evidence of Chlamydia Pneumoniae Infection RETINA CONGRESS 2002 SCIENTIFIC PAPER ABSTRACTS 138-139).

Chlamydia pneumoniae is a member of the Chlamydia genus, whose members have a unique, biphasic life cycle that occurs entirely within eukaryotic cells (see, Kalayoglu (2002) supra; Peeling & Brunham (1996) ENMRG. INFECT. DIS. 2: 307-319). Inside the host cell, the pathogen exists as either an infectious but metabolically inert elementary body (EB), or a non-infectious but metabolically active reticulate body (RB). The EB enters the host cell and prevents fusion of the phagosome with lysosomes. The EB thus survives within this inclusion and differentiates into the metabolically active RB, which multiply by binary fission and re-differentiate into EBs. Subsequent cell lysis and escape of infectious EBs into the external milieu completes the life cycle. In addition, it is understood that Chlamydiae may adopt a non-infectious, non-metabolically active persistent form under certain stress conditions. For example, exposure of infected cells to the T-cell cytokine interferon gamma (IFN-γ) induces formation of persistent Chlamydiae, characterized in part by aberrant morphology, inhibited expression of immunodominant outer envelope complex proteins, and enhanced expression of the inflammatory heat shock protein 60 (cHsp60) (Beatty et al. (1994) MICROBIOL. REV. 58: 686-699, and Beatty et al. (1993) PROC. NATL. ACAD. SCI. USA 90: 3998-4002). Removal of IFN-γ permits re-differentiation of persistent Chlamydiae into infectious EBs and completion of a normal life cycle (Beatty (1993) supra). Such differential expression of virulence determinants may aid the organism in evading an immune response while maintaining inflammation in the local milieu. In vivo, the capacity of chlamydiae to adopt a transient, persistent state may explain why the hallmark of Chlamydial infection is chronic, inflammatory disease (Ward (1995) APMIS 103, 769-796). Accordingly, one or more of the different forms Chlamydia pneumoniae may be detected as a way to determine whether an individual has been infected by Chlamydia pneumoniae.

Alternatively, rather than detecting one of the different forms of the viable organism, it may be helpful to detect as an indicator of Chlamydia pneumoniae infection a Chlamydia pneumoniae specific agent. Chlamydia pneumoniae specific agents include, for example, a Chlamydia pneumoniae specific nucleic acid molecule (for example, DNA or RNA), a Chlamydia pneumoniae specific protein or peptide sequence, a Chlamydia pneumoniae specific saccharide, a Chlamydia pneumoniae specific polysaccharide, a Chlamydia pneumoniae specific lipopolysaccharide, or a Chlamydia pneumoniae specific surface antigen or antigenic determinant.

Alternatively, useful Chlamydia pneumoniae specific agents also include molecules created, induced or synthesized by the host organism in response to a Chlamydia pneumoniae infection. Exemplary host created or synthesized molecules include antibodies (for example, antibodies to Chlamydial outer membrane proteins and lipopolysaccharide) and acute-phase proteins such as C-reactive protein (CRP). A host antibody useful as a marker of Chlamydia pneumoniae infection includes an antibody that binds specifically to an EB, Chlamydial heat shock protein 60, Chlamydial major outer membrane protein (MOMP), and Chlamydial lipopolysaccharide (LPS).

In another aspect, the invention provides a kit comprising an agent for detecting Chlamydia pneumoniae or a Chlamydia pneumoniae specific agent and instructions directing a user on how to detect Chlamydia pneumoniae or a Chlamydia pneumoniae specific agent in a sample to determine if a mammal is at risk of developing, or has, a chronic intraocular disorder. The kit may also comprise a receptacle for receiving a tissue or fluid sample from the mammal. The agents may include, for example, an anti-Chlamydia pneumoniae specific antibody, or a Chlamydia pneumonia specific agent for use in an immunological assay, optionally together with one or more reagents for performing an immunological reaction. Alternatively, agents may include, for example, a nucleic acid sequence that hybridizes specifically to a Chlamydia pneumoniae specific nucleic acid, optionally with one or more reagents for performing a nucleic acid amplification and/or detection type assay.

In yet another aspect, the invention provides a method of preventing, slowing or stopping the progression of a chronic intraocular disorder, for example, age-related macular degeneration, uveitis syndromes (for example, chronic iridocyclitis and chronic endophthalmitis), choroidopathies (for example, “White-dot” syndromes including, but not limited to, acute multifocal posterior placoid), ocular glaucomas (for example, inflammatory glaucomas), retinopathies (for example, cystoid macular edema, central serous choroidopathy and presumed ocular histoplasmosis syndrome), retinal vascular disease (for example, diabetic retinopathy, Coat's disease, and retinal arterial macroaneurysm) and cataracts. The method comprises administering to a mammal, for example, a primate, for example, a human, suspected of having or developing a chronic intraocular disorder an amount of an anti-Chlamydia pneumoniae agent sufficient to kill or inactivate Chlamydia pneumonia so as to prevent, slow or stop the progression of the disorder.

It is contemplated that a variety of anti-Chlamydia pneumoniae agents may be administered to the mammal and may include, for example, an anti-Chlamydia pneumoniae antibiotic, vaccine, antibody, Chlamydial LPS-binding protein or Chlamydial LPS-antagonist. The agent of interest may be administered prophylactically, for example, after a mammal has been identified as having been infected with Chlamydia pneumoniae but before the onset or the diagnosable onset of a chronic intraocular disorder. Such treatment may prevent the onset of the disorder, thereby minimizing or eliminating vision loss which otherwise may occur as a result of the disorder. Alternatively, the agent may be administered therapeutically, for example, after a mammal has been diagnosed as having the chronic intraocular disorder. Such treatment may slow, stop or even reverse the progression of the disorder thereby minimizing, eliminating or restoring vision loss which otherwise may occur as a result of the disorder.

Depending upon the agent to be administered, it may be administered via standard procedures and at standard dosages known and used in the art. For example, the agent may be administered systemically or locally. Systemic modes of administration include, for example, parenteral administration, intravascular administration, and intramuscular administration. Alternatively, the agent may be applied locally, for example, via localized delivery to the eye or orbital socket by methods including, but not limited to, intravitreal delivery, transscleral delivery, peribulbar injection, retrobulbar injection, and sub-tenons injection. In addition, it is contemplated that the agent may be administered as one or more boluses or via continuous delivery. In addition, it is contemplated that a plurality of different anti-Chlamydia pneumoniae agents may be administered to the individual, which may be administered simultaneously or sequentially.

The foregoing aspects and embodiments of the invention may be more fully understood by reference to the following figures, detailed description and claims.

### BRIEF DESCRIPTION OF THE DRAWINGS

The objects and features of the invention may be more fully understood by reference to the drawings described below in which:

FIG. 1 is a chart showing the levels of anti-Chlamydia pneumoniae elementary body antibody titers in individuals with age-related macular degeneration (filled circles) versus individuals without age-related macular degeneration (unfilled circles), where mean values are shown by a horizontal line;

FIG. 2 is a bar chart showing the percentage of individuals with (filled bars) or without (unfilled bars) age-related macular degeneration and the respective titers of anti-Chlamydia pneumoniae elementary body antibodies as separated into quintiles, where the quintiles were determined by partitioning the optical density readouts into fifths;

FIGS. 3a and 3b show the amount of vascular endothelial growth factor (VEGF) produced by monocyte-derived macrophages infected with Chlamydia pneumonia. FIG. 3a is a bar chart showing the amount of VEGF (pg/ml) produced by monocyte-derived macrophages infected with different doses of Chlamydia pneumoniae. FIG. 3b is a graph showing the amount of VEGF (pg/ml) produced by monocyte-derived macrophages infected with Chlamydia pneumoniae (filled circles) or uninfected with Chlamydia pneumoniae (unfilled circles) over time;

FIGS. 4a and 4b show the amount of inteleukin-8 (IL-8) produced by retinal pigment epithelial (RPE) cells infected with Chlamydia pneumonia. FIG. 4a is a bar chart showing the amount of IL-8 (pg/ml) produced by RPE cells infected with different doses of Chlamydia pneumoniae. FIG. 4b is a graph showing the amount of IL-8 (pg/ml) produced by RPE cells infected with Chlamydia pneumoniae (filled circles) or uninfected with Chlamydia pneumoniae (unfilled circles) over time; and

FIGS. 5a and 5b show the amount of monocyte chemotactic protein 1 (MCP-1) produced by RPE cells infected with Chlamydia pneumoniae. FIG. 5a is a bar chart showing the amount of MCP-1 (pg/ml) produced by RPE cells infected with different doses of Chlamydia pneumoniae. FIG. 5b is a graph showing the amount of MCP-1 (pg/ml) produced by RPE cells infected with Chlamydia pneumoniae (filled circles) or uninfected with Chlamydia pneumoniae (unfilled circles) over time.

### DETAILED DESCRIPTION OF THE INVENTION

Chlamydia pneumoniae infection has been shown to be a risk factor associated with the development of certain chronic intraocular disorders. As a result, the invention provides methods of determining whether an individual is at risk of developing or has a chronic intraocular disorder. In addition, by treating to remove this treatable risk factor, the invention provides methods of preventing the onset, and, under certain circumstances, the development of certain chronic ocular disorders.

In one aspect, the invention provides a method of determining whether a mammal is at risk of developing or has a chronic intraocular disorder. The method comprises detecting in a sample harvested from the mammal, for example, a primate, for example, a human, the presence of Chlamydia pneumoniae or a Chlamydia pneumoniae specific agent, the presence and/or concentration of which is indicative that the mammal is at risk of developing or has the chronic intraocular disorder.

As used herein, the term “chronic intraocular disorder” is understood to mean an ocular disorder, the pathogenesis of which evolves over weeks, months or years. Examples of chronic intraocular disorders include, without limitation, age-related macular degeneration, uveitis syndromes (for example, chronic iridocyclitis and chronic endophthalmitis), choroidopathies (for example, “White-dot” syndromes including, but not limited to, acute multifocal posterior placoid), ocular glaucomas (for example, inflammatory glaucomas), retinopathies (for example, cystoid macular edema, central serous choroidopathy and presumed ocular histoplasmosis syndrome), retinal vascular disease (for example, diabetic retinopathy, Coat's disease and retinal arterial macroaneurysm) and cataracts. Non-arteritic anterior ischemic optic neuropathy, acute anterior uveitis, branch retinal vein occlusion and chronic conjunctivitis are specifically excluded from being chronic intraocular disorders.

In one approach, the method involves detecting one or more of the various forms of Chlamydia pneumoniae, including, for example, elementary bodies (EB), reticulate bodies (RB), and persistent bodies (PB). In another approach, the method involves detecting the presence of a Chlamydia pneumoniae specific agent. As used herein, the term “Chlamydia pneumoniae specific agent” is understood to mean (i) one or more molecules, at least a portion of which is specific to the Chlamydia pneumoniae organism, for example, a protein, peptide, nucleic acid (for example, DNA or RNA), lipid, saccharide, polysaccharide, lipopolysaccharide, surface antigen or antigenic determinant, and a secreted Chlamydial product, or a combination thereof, and/or (ii) one or more molecules that are induced and/or produced in a host mammal following infection with Chlamydia pneumoniae, for example, an anti-Chlamydia pneumoniae specific antibody (for example, antibodies that specifically bind Chlamydia pneumoniae EB, Chlamydia pneumoniae heat shock protein 60, Chlamydial major outer membrane protein (MOMP), Chlamydial LPS, Chlamydial outer membrane proteins and Chlamydial lipopolysaccharides), Chlamydial acute phase proteins such as C-reactive protein (CRP), or a combination thereof.

The Chlamydia pneumoniae, the Chlamydia pneumoniae specific agent, or a combination thereof, may be detected in a wide variety of samples that can be harvested from the mammal. For example, the Chlamydia pneumoniae and/or Chlamydia pneumoniae specific agent may be detected in a body fluid sample selected from the group consisting of blood, serum, plasma, urine, lacrimal fluid, vitreous, aqueous, and synovial fluid. In addition, the Chlamydia pneumoniae and/or Chlamydia pneumoniae specific agent may be detected in a tissue sample selected from the group consisting of skin, conjunctiva, cornea, sclera, uvea, retina, choroid, neovascular tissue, and optic nerve.

A variety of methods may be used to detect the presence of Chlamydia pneumoniae and/or a Chlamydia pneumoniae specific agent in a sample. Exemplary protein- and nucleic acid-based assays are discussed in more detail below.

### I. METHODS FOR DETECTING CHLAMYDIA PNEUMONIAE AND CHLAMYDIA PNEUMONIAE SPECIFIC AGENTS

The presence and/or amount of Chlamydia pneumoniae and/or Chlamydia pneumoniae specific agents may be determined using a variety of techniques known and used in the art (see for example, U.S. Pat. No. 6,579,854). A variety of exemplary techniques are described in more detail below.

(i) Chlamydia Pneumoniae

The presence and amount of the Chlamydia pneumoniae organism may be determined by various protocols. In one exemplary technique, the sample to be tested for the presence of Chlamydia pneumoniae is added to plated and cultured human epithelial cells, for example, Hep-2 cells (an epithelial cell line). Following incubation of the cells, an FITC-conjugated monoclonal antibody that specifically binds Chlamydial MOMP or Chlamydial LPS is added to the cells. Following incubation and the removal of unbound antibody via washing with wash solution, the cells are viewed under an epifluorescent microscope and the number of Chlamydial inclusions counted. The multiplicity of infection (MOI) can be calculated by dividing the number of inclusions by the number of cells. The MOI conveys how many infectious Chlamydia were in the original sample.

(ii) Chlamydia Pneumoniae Specific Agents

It is contemplated that the Chlamydia pneumoniae specific agents include components originating from Chlamydia pneumoniae itself, for example, a Chlamydia pneumoniae specific nucleic acid, protein, peptide, lipid, saccharide, polysaccharide, lipopolysaccharide, and a secreted Chlamydial product, or may include a molecule induced or synthesized in the host following a Chlamydia pneumoniae infection, for example, an anti-Chlamydial antibody. The methods typically comprise the steps of detecting, by some means, the presence of one or more proteins, peptides, or nucleic acids encoding such proteins peptide, lipid, saccharide, polysaccharide, lipopolysaccharide in a tissue or body fluid sample. The accuracy and/or reliability of the method for detecting the presence of an ongoing or prior infection with Chlamydia pneumoniae may be further enhanced by detecting the presence of a plurality of Chlamydia pneumoniae specific agents, for example, a plurality of proteins and/or nucleic acids in a preselected tissue or body fluid sample. The detection assays may comprise one or more of the protocols described hereinbelow.

Protein-Based Assays

Chlamydia pneumoniae specific marker proteins or peptides may be detected in a sample of interest using any of a variety of techniques known in the art, a subset of which are discussed below. It is contemplated that the existence of host induced and/or host synthesized marker proteins or peptides that are stimulated by a Chlamydia pneumoniae infection may also be detected using the same or similar techniques.

In one approach, for example, the marker protein or peptide may be detected using a binding moiety capable of specifically binding the marker protein or peptide. The binding moiety may comprise, for example, a member of a ligand-receptor pair, i.e., a pair of molecules capable of having a specific binding interaction. The binding moiety may comprise, for example, a member of a specific binding pair, such as antibody-antigen, enzyme-substrate, protein-nucleic acid, protein-protein, or other specific binding pair known in the art. Binding proteins may be designed which have enhanced affinity for a marker protein or peptide. Optionally, the binding moiety may be linked with a detectable label, such as an enzymatic, fluorescent, radioactive, phosphorescent, colored particle label or spin label. The labeled complex may be detected, e.g., visually or with the aid of a spectrophotometer or other detector.

A wide variety of different labels may be employed in the assays discussed herein. The labeled reagents may be provided in solution or coupled to an insoluble support, depending on the design of the assay. The various conjugates may be joined covalently or noncovalently, directly or indirectly. When bonded covalently, the particular linkage group will depend upon the nature of the two moieties to be bonded. A large number of linking groups and methods for linking two moieties are known in the art. Broadly, the labels may be divided into the following general categories: chromogens, catalyzed reactions, chemiluminescence, radioactive labels, and colloidal-sized colored particles. The chromogens include compounds which absorb light in a distinctive range so that a color may be observed, or emit light when irradiated with light of a particular wavelength or wavelength range, for example, fluorescers. Both enzymatic and nonenzymatic catalysts may be employed. In choosing an enzyme, there will be many considerations including the stability of the enzyme, whether it is normally present in samples of the type for which the assay is designed, the nature of the substrate, and the effect if any of conjugation on the enzyme's properties. Potentially useful enzyme labels include oxiodoreductases, transferases, hydrolases, lyases, isomerases, ligases, or synthetases. Interrelated enzyme systems may also be used. A chemiluminescent label involves a compound that becomes electronically excited by a chemical reaction and may then emit light that serves as a detectable signal or donates energy to a fluorescent acceptor. Radioactive labels include various radioisotopes found in common use such as the unstable forms of hydrogen, iodine, phosphorus or the like. Colloidal-sized colored particles involve material such as colloidal gold that, in aggregate, form a visually detectable distinctive spot corresponding to the site of a substance to be detected. Additional information on labeling technology is disclosed, for example, in U.S. Pat. No. 4,366,241.

The marker protein or peptide may be detected using any of a wide range of, for example, immunoassay techniques available in the art. For example, the skilled artisan may employ a sandwich immunoassay format to detect the presence of a marker protein or peptide in a sample. Alternatively, the skilled artisan may use conventional immunohistochemical procedures for detecting the presence of a marker protein or peptide in: tissue sample using one or more labeled binding proteins.

In a sandwich immunoassay, for example, two antibodies capable of binding the marker protein generally are used, e.g., one immobilized onto a solid support, and one free in solution and labeled with a detectable chemical moiety. Examples of chemical moieties that may be used for the second antibody include radioisotopes, fluorescent compounds, spin labels, colored particles such as colloidal gold and colored latex, and enzymes or other molecules that generate colored or electrochemically active products when exposed to a reactant or enzyme substrate. When a sample containing the marker protein is placed in this system, the marker protein binds to both the immobilized antibody and the labeled antibody, to form a “sandwich” immune complex on the surface of the support. The complexed marker protein is detected by washing away non-bound sample components and excess labeled antibody, and measuring the amount of labeled antibody specifically bound to the solid support. Alternatively, the antibody free in solution, which can be labeled with a chemical moiety, for example, a hapten, may be detected by a third antibody labeled with a detectable moiety which binds the free antibody or, for example, the hapten coupled thereto.

Similarly, a host specific protein or peptide synthesized in response to a Chlamydia pneumoniae infection, for example, an anti-Chlamydia pneumoniae antibody, may also be detected using any of a wide range of immunoassay techniques known in the art. For example, the skilled artisan may use a variation of the sandwich approach for detecting the presence of a specific host antibody in a sample. By way of example, a ligand (for example, a Chlamydial EB) known to be bound by a host specific antibody of interest (for example, a host anti-EB antibody) is immobilized on a solid support. When a sample containing the host antibody is applied to the solid support, the host antibody binds specifically to its cognate ligand pre-immobilized on the solid support. Unbound components are removed by washing the solid support. The presence of the bound host antibody can then be detected using a labeled binding protein, for example, a goat or rabbit anti-human antibody, that specifically binds the bound host antibody. The residual unbound labeled binding protein then is removed by washing, and the specifically bound labeled binding protein, if any, then is detected, for example, visually or via a mechanical detector. In the absence of host antibody, little or no labeled binding protein binds the solid support. On the other hand, if host antibody is present in the sample, the labeled binding protein binds to the solid support. The amount of bound labeled antibody is indicative of whether there are host specific antibodies in the sample of interest.

As another type of approach, immunoprecipitation assays may be useful in the practice of the invention. Immunoprecipitation techniques generally are performed in solution and rely on the formation of detectable antigen-antibody complexes. The formation of these complexes may be associated with changes in the optical properties of the solution. These changes can, in turn, be detected with a suitable optical detector. In a nephelometric immunoassay, for example, a photometer is used to measure the reflection or scatter of light by immunoprecipitates towards a light detector. The immunoprecipitates can be aggregates of a Chlamydia pneumoniae specific protein and a specific binding protein for that protein. The amount of light scattered by the immunoprecipitates is directly proportional to the number of immunoprecipitates present, which typically increases as the reaction proceeds. This proportionally permits a quantitative determination of analyte concentration.

In turbidimetric immunoassays, an attenuation or reduction of light energy passing through a liquid medium containing immunoprecipitates is measured by a light detector placed in the light path. The light energy reduction can be caused by reflection, scatter, and absorption of the incident light by the immunoprecipitates. The amount of light reduction caused by the immunoprecipitate is directly proportional to the number of immunoprecipitates present, permitting a quantitative determination of analyte concentration. Nephelometric and turbidimetric immunoassays are further described in U.S. Pat. No. 5,583,055 and the references cited therein.

The presence of a Chlamydia pneumoniae specific protein can also be detected by immunoprecipitation where at least one of the components of the resulting complex bears a radioactive label. The radiolabel may be coupled to an antibody that specifically binds the Chlamydia pneumoniae specific protein, or may be coupled to another binding partner or moiety that binds to the antibody. After forming complexes of the antibody and the Chlamydia pneumoniae specific protein, the precipitated complexes are separated from the “free” antibody. The amount of radioactivity in the precipitated complexes then is counted by standard methods (e.g., with a scintillation counter, a gamma counter, or a Geiger counter). Generally, the amount of radioactivity detected is directly proportional to the amount of the Chlamydia pneumoniae specific protein present in the sample being analyzed. Similar immunopreciptation assays may also be employed to detect the presence of host antibodies in the sample of interest.

Each of the above methodologies can be highly sensitive. A detailed review of immunological assay design, theory and protocols can be found in numerous texts in the art, including Butt, ed., (1984) PRACTICAL IMMUNOLOGY, Marcel Dekker, New York; Harlow et al. eds. (1988) ANTIBODIES, A LABORATORY APPROACH, Cold Spring Harbor Laboratory; and Diamandis et al., eds. (1996) IMMUNOASSAY, Academic Press, Boston.

In general, immunoassay design considerations include the preparation of antibodies (e.g., monoclonal or polyclonal antibodies) having sufficiently high binding specificity for the marker protein to form a complex that can be distinguished reliably from products of nonspecific interactions. As used herein, the term “antibody” is understood to mean binding proteins, for example, antibodies or other proteins comprising an immunoglobulin variable region-like binding domain, having the appropriate binding affinities and specificities for the marker protein. The higher the antibody binding specificity, the lower the marker protein concentration that can be detected. As used herein, the terms “specific binding” or “binding specifically” are understood to mean that the binding moiety, for example, a binding protein has a binding affinity for the target protein of greater than about 10⁵ M⁻¹, more preferably greater than about 10⁷ M⁻¹.

Antibodies to an isolated target Chlamydia pneumoniae specific marker protein which are useful in assays for detecting a Chlamydial infection in an individual may be generated using standard immunological procedures known and used in the art. See, for example, Butt, ed., (1994) supra. Briefly, an isolated Chlamydia pneumoniae specific protein is used to raise antibodies in a xenogeneic host, such as a mouse, goat or other suitable mammal. The marker protein is combined with a suitable adjuvant capable of enhancing antibody production in the host, and is injected into the host, for example, by intraperitoneal administration. Any adjuvant suitable for stimulating the host's immune response may be used. A commonly used adjuvant is Freund's complete adjuvant. Where multiple antigen injections are desired, the subsequent injections may comprise the antigen in combination with an incomplete adjuvant (e.g., a cell-free emulsion). Polyclonal antibodies may be isolated from the antibody-producing host by extracting serum containing antibodies to the marker protein of interest. Monoclonal antibodies may be produced by isolating host cells that produce the desired antibody, fusing these cells with myeloma cells using standard procedures known in the immunology art, and screening for hybrid cells (hybridomas) that react specifically with the marker protein and have the desired binding affinity.

Antibody binding domains also may be produced biosynthetically and the amino acid sequence of the binding domain manipulated to enhance binding affinity with a preferred epitope on the target protein. Specific antibody methodologies are well understood and described in the literature. A more detailed description of their preparation can be found, for example, in Butt, ed., (1984) supra. In addition, genetically engineered biosynthetic antibody binding sites, also known in the art as BABS or sFv's, may be used in the practice of the instant invention. Methods for making and using BABS comprising (i) non-covalently associated or disulfide bonded synthetic V_{H} and V_{L} dimers, (ii) covalently linked V_{H}-V_{L} single chain binding sites, (iii) individual V_{H} or V_{L} domains, or (iv) single chain antibody binding sites are disclosed, for example, in U.S. Pat. Nos. 5,091,513; 5,132,405; 4,704,692; and 4,946,778. Furthermore, BABS having requisite specificity for marker proteins of interest can be derived by phage antibody cloning from combinatorial gene libraries (see, for example, Clackson et al. (1991) NATURE 352: 624-628, and U.S. Pat. No. 5,837,500). Briefly, phage each expressing on their coat surfaces, BABS having immunoglobulin variable regions encoded by variable region gene sequences derived from mice pre-immunized with an isolated Chlamydia pneumoniae specific protein, or fragments thereof, are screened for binding activity against immobilized marker protein. Phage which bind to the immobilized marker protein are harvested and the gene encoding the BABS sequenced. The resulting nucleic acid sequences encoding the BABS of interest then may be expressed in conventional expression systems to produce the BABS protein.

Furthermore, a sandwich immunoassay for detecting a Chlamydia pneumoniae specific protein may be implemented in a lateral flow device. Lateral flow immunochromatographic test devices are described, for example, in U.S. Pat. Nos. 5,622,871; 5,714,389; and 5,591,645. Generally, the device includes an absorbent portion for receiving a liquid sample to be tested, such as serum or urine, and an absorbent test strip. A specific binding moiety, such as an antibody, that specifically binds to the Chlamydia pneumoniae specific protein is immobilized in a defined portion of the test strip. Following application to the absorbent portion, the liquid sample flows by wicking or capillary action from the absorbent portion to the test strip, and along the test strip past the specific binding moiety, which binds and immobilizes the Chlamydia pneumoniae specific protein. A second, labeled, mobile specific binding moiety is used to detect the presence of the immobilized Chlamydia pneumoniae specific protein. The second, labeled specific binding moiety may be mixed with the sample prior to application of the sample to the device, may be added to the device after the application of the sample to the device, or may be incorporated into the device in such a way that it becomes solubilized in the liquid sample as the sample flows through the device and before the sample reaches the immobilized specific binding moiety.

Marker proteins may also be detected using gel electrophoresis techniques, such as one-dimensional or two-dimensional gel electrophoresis. In two-dimensional gel electrophoresis, the proteins are separated first in a pH gradient gel according to their isoelectric point. The resulting gel then is placed on a second polyacrylamide gel, and the proteins separated according to their molecular weight (see, for example, O'Farrell (1975) J. BIOL. CHEM. 250: 4007-4021). One or more marker proteins may be detected by first isolating proteins from a sample obtained from an individual suspected of having been infected with Chlamydia pneumoniae, and then separating the proteins under the same or similar conditions, as proteins isolated from either individuals diagnosed as having been infected with Chlamydia pneumoniae (positive control) or having been diagnosed as being uninfected (negative control). The standard gel pattern may be stored in, and retrieved from, an electronic database of electrophoresis patterns. The presence of a Chlamydia pneumoniae specific protein in the two-dimensional gel as detected, for example, by conventional dye staining or immunobathing, provides an indication that the sample being tested was taken from a person having been infected with Chlamydia pneumoniae. As with the other detection assays described herein, the detection of two or more proteins, for example, in the two-dimensional gel electrophoresis pattern further enhances the accuracy of the assay. The presence of a plurality, for example, two, three, four, five or more Chlamydia pneumoniae specific proteins on a two-dimensional gel provides an even stronger indication that the individual from whom the sample was derived has been infected with Chlamydia pneumoniae.

Mass spectrometry may also be used to detect a marker protein in a sample of interest. Preferred mass spectrometry methods include MALDI-TOF mass spectrometry and MALDI-TOF using derivatized chip surfaces (SELDI). Useful mass spectrometry methods for detecting a marker protein are described, for example, in U.S. Pat. Nos. 5,719,060; 5,894,063; 6,124,137; 6,207,370; 6,225,047; 6,281,493; and 6,322,970. In these methods, the presence and/or amount of a particular marker protein or a plurality of different marker proteins in a separation profile can be monitored. In such approaches, the separation profile of a marker protein or proteins derived from a test patient of unknown disposition may be compared against the separation profile of the marker protein or proteins derived from a control sample (for example, a negative control where an individual has been confirmed to have not been infected with Chlamydia pneumoniae or a positive control where individual(s) is or are have been confirmed as having been infected with Chlamydia pneumoniae specific protein). The amounts of one or more of the marker proteins in the test sample relative to the amount of the same or similar proteins in the control sample can be an indicator of whether the individual providing the test sample may have been infected with Chlamydia pneumoniae. For example, a result in which the amount of a particular marker protein in the separation profile from a test individual is less than or equal to the amount of marker protein in a negative control sample is indicative that the test individual has not been infected with Chlamydia pneumoniae. In contrast, a result in which the amount of a particular marker protein in the separation profile from a test individual is greater than the amount of the marker protein in a positive control sample is indicative that the test individual may have been infected with Chlamydia pneumoniae.

The detection methods described herein may be used in combination with each other, with other detection methods, and/or with one or more purification methods to reduce the complexity of a biological sample. Thus, for example, proteins isolated by gel electrophoresis could be probed with an antibody that specifically binds the marker protein, or could be assayed by mass spectrometry. Similarly, a biological sample may be subjected to biochemical fractionation prior to analysis by mass spectrometry or by other techniques such as gel electrophoresis and/or immunoassays. Further details for such procedures may be found, for example, in International application serial number PCT/US00/31492.

Nucleic Acid-Based Assays

The presence of a Clamydia pneumoniae infection in an individual may also be determined by detecting, in a tissue or body fluid sample, a Chlamydia pneumoniae specific nucleic acid sequence, for example, a nucleic acid sequence, at least a portion of which is unique to Chlamydia pneumoniae.

A target Chlamydia pneumoniae specific nucleic acid molecule may be detected using a labeled binding moiety capable of specifically binding the target nucleic acid. The binding moiety may comprise, for example, a protein, a nucleic acid or a peptidyl nucleic acid. Additionally, a target nucleic acid, such as an mRNA encoding a Chlamydia pneumoniae specific protein or protein fragment, may be detected by conducting, for example, a Northern blot analysis using labeled oligonucleotides, e.g., nucleic acid fragments complementary to and capable of hybridizing specifically with at least a portion of a target nucleic acid.

More specifically, gene probes comprising complementary RNA or, preferably, DNA to the Chlamydia pneumoniae specific nucleic acid sequences may be produced using established recombinant techniques or oligonucleotide synthesis techniques. The probes hybridize with complementary nucleic acid sequences presented in the test specimen, and can provide exquisite specificity. A short, well-defined probe, coding for a single unique sequence is most precise and preferred. Larger probes are generally less specific. While an oligonucleotide of any length may hybridize to an mRNA transcript, oligonucleotides typically within the range of 8-100 nucleotides, preferably within the range of 15-50 nucleotides, are envisioned to be most useful in standard hybridization assays. Choices of probe length and sequence allow one to choose the degree of specificity desired. Hybridization is carried out at from 50° to 65° C. in a high salt buffer solution, formamide or other agents to set the degree of complementarity required. Furthermore, the state of the art is such that probes can be manufactured to recognize essentially any DNA or RNA sequence. More preferably, the probes hybridize to the Chlamydia pneumoniae specific nucleic acid sequences or sequences complementary thereto under stringent hybridization and washing conditions. For additional particulars, see, for example, Berger et al. (1987) GUIDE TO MOLECULAR TECHNIQUES (METHODS OF ENZYMOLOGY, Vol. 152).

The probes may be labeled with a variety of different labeling moieties using a variety of different techniques. For example, one method of in vitro labeling of nucleotide probes involves nick translation where an unlabeled DNA probe is nicked with an endonuclease to produce free 3′ hydroxyl termini within either strand of the double-stranded fragment. Simultaneously, an exonuclease removes the nucleotide residue from the 5′phosphoryl side of the nick. The sequence of replacement nucleotides is determined by the sequence of the opposite strand of the duplex. Thus, if labeled nucleotides are supplied, DNA polymerase fills in the nick with the labeled nucleotides. Using this well-known technique, up to 50% of the molecule can be labeled. For smaller probes, known methods involving 3′end labeling may be used. Furthermore, there are currently commercially available methods of labeling DNA with fluorescent molecules, catalysts, enzymes, or chemiluminescent materials. Biotin labeling kits are commercially available (Enzo Biochem, Inc.) under the trademark Bio-Probe. This type of system permits the probe to be coupled to avidin which in turn is labeled with, for example, a fluorescent molecule, enzyme, antibody, etc. For further disclosure regarding probe construction and technology, see, for example, Sambrook et al. (1989) supra, or Wu et al. (1997) METHODS IN GENE BIOTECHNOLOGY, CRC Press, New York.

The oligonucleotide selected for hybridizing to the target nucleic acid, whether synthesized chemically or by recombinant DNA methodologies, is isolated and purified using standard techniques and then preferably labeled (e.g., with ³⁵S or ³²P) using standard labeling protocols. A sample containing the target nucleic acid then is run on an electrophoresis gel, the fractionated nucleic acids then are transferred to a nitrocellulose or nylon membrane and the labeled oligonucleotide exposed to the filter under stringent hybridizing conditions, e.g., 50% formamide, 5×SSPE, 2× Denhardt's solution, 0.1% SDS at 42° C., as described in Sambrook et al. (1989) supra. The filter may then be washed to remove unbound, or non specifically bound labeled oligonucleotide using, for example, 2×SSPE, 0.1% SDS at 68° C., and more preferably using 0.1×SSPE, 0.1% SDS at 68° C. Other useful procedures known in the art include solution hybridization, and dot and slot RNA hybridization. Optionally, the amount of the target nucleic acid present in a sample then is quantitated by measuring the radioactivity of hybridized fragments, using standard procedures known in the art.

In addition, the presence and/or amount of a Chlamydia pneumoniae specific nucleic acid may be determined using a combination of appropriate oligonucleotide primers, i.e., more than one primer, using standard polymerase chain reaction (PCR) procedures, for example, by real-time quantitative PCR. Conventional PCR based assays are discussed, for example, in Ifles et al (1990) PCR PROTOCOLS: A GUIDE TO METHODS AND APPLICATIONS, Academic Press and Innes et al. (1995) PCR Strategies, Academic Press, San Diego, Calif.

### II. METHODS FOR PREVENTING THE ONSET OF OR SLOWING THE DEVELOPMENT A CHRONIC INTRAOCULAR DISORDER

Once identified as having been infected with Chlamydia pneumoniae, the infected individual is at risk of developing a chronic intraocular disorder, for example, age-related macular degeneration. Accordingly, the individual may be monitored on a regular basis using standard methodologies for the onset of the chronic intraocular disorder. This approach may facilitate early intervention and treatment of the disorder, which otherwise may progress until substantial irreversible vision loss has occurred. Similarly, the individual may be treated prophylactically with an amount of an anti-Chlamydia pneumoniae agent sufficient to kill or inactivate Chlamydia pneumoniae (also referred to as an effective amount) to treat this treatable risk factor thereby to prevent or slow down the onset of the ocular disorder.

Alternatively, if the individual has been diagnosed as both having been infected with Chlamydiae pneumoniae and having the chronic intraocular disorder, then the individual may be treated therapeutically with an effective amount of an anti-Chlamydiae pneumoniae agent, for example, an antibiotic or anti-Chlamydial vaccine, so as to slow down, stop or even reverse the chronic intraocular disorder. Which ever approach is used, the objective is to prevent, minimize, slow down or even reverse vision loss in the individual.

It is contemplated that a variety of anti-Chlamydia pneumoniae agents may be useful in the practice of this aspect of the invention. Such agents include, for example, one or more antibiotics, vaccines, Chlamydial CPS-binding proteins, Chlamydia heat shock protein 60 antagonists, or Chlamydial LPS-antagonists, or a combination thereof, that kill, inactivate, or otherwise prevent infection by Chlamydia pneumoniae. In addition, it is further contemplated that a combination of agents which are effective against different phases of the Chlamydial life cycle may also be useful in the practice of the invention. For example, useful agents include agents which are effective at inhibiting against the cryptic phase, the elementary body phase or the replicating phase of the Chlamydial life cycle.

Agents which inhibit different phases of the Chlamydial life cycle are known in the art. For example, disulfide reducing agents such as 2,3-dimercaptosuccinic acid (DMSA); beta-lactam agents (for example, penicillins, penicillin G, ampicillin and amoxicillin, which produce penicillamine as a degradation product), cycloserine, dithiotreitol, mercaptoethylamine (e.g., mesna, cysteiamine, dimercaptol), N-acetylcysteine, tiopronin, and glutathione are known to be effective against the elementary body phase. Nitroaromatic compounds such as metronidazole, tinidazole, bamidazole, benznidazole, flunidazole, ipronidazole, misonidazole, moxnidazole, ronidazole, sulnidazole, and their metabolites, analogs and derivatives thereof, are known to be effective against the cryptic phase. Quinolones (for example, Ofloxacin), fluoroquinolones (for example, Levofloxacin, Trovafloxacin, Sparfloxacin, Norfloxacin, Lomefloxacin, Cinoxacin, Enoxacin, Nalidixic Acid, Fleroxacin and Ciprofloxacin), sulfonamides (for example, Sulfamethoxazole and Trimethoprin), azalides (for example, Azithromycin), macrolides (for example, Erythromycin and Clarithromycin), lincosamides (for example, Lincomycin and Clindamycin), tetracyclines (for example, Tetracycline, Doxycycline, Minocycline, Methacycline, and Oxytetracycline) and rifamycins (for example, Rifampin and Rifabutin) are known to be effective against the replicating phase of Chlamydia. In addition, another class of anti-Chlamydial agent that is effective against the replicating and cryptic stationary phases of Chlamydia includes ethambutol and isonicotinic acid congeners.

Exemplary antibiotics include, for example, Erythromycin (available from Eli Lilly Co., Indianapolis, Ind.), Azithromycin (available from Pfizer Inc., Providence, R.I.), Tetracycline (available from Idexx Laboratories Inc., Greensboro, N.C.), Doxycycline (available from Pfizer Inc., Providence, R.I.), Clarithromycin (available from Abbott Laboratories, Abbott Park, Ill.), Quinolones, Fluoroquinolones including, but not limited to, Garenoxacin (available from Bristol-Meyers Squibb Corp., New York, N.Y.), Levofloxacin (available from Janssen-Cilag, Titusville, N.J.), Ciprofloxacin (available from Sigma Chemicals,), Gatifloxacin (available from Bristol-Meyers Squibb Corp., New York, N.Y.), Moxifloxacin (available from Bayer, Inc., Morristown, N.J.) and Gemifloxacin (available from Bristol-Meyers Squibb Corp., New York, N.Y.), and Benzaxinorifamycin (Activbiotics, Cambridge, Mass.).

Screening Assays

The invention provides methods for identifying compounds that bind to Chlamydia pneumoniae or a Chlamydia pneumoniae specific agent such as a protein, for example, Chlamydia pneumoniae heat shock protein 60. Candidate compounds that can be screened in accordance with the invention include, but are not limited to, polypeptides, oligopeptides, antibodies, and monomeric organic compounds, i.e., “small molecules.” The skilled artisan can identify one or more compounds that inhibit Chlamydial infectivity of a cell. The compounds can be identified by, for example, screening compound libraries for molecules that bind heat shock protein 60 of Chlamydia pneumonia and then determining if the compound can inhibit Chlamydial infectivity of the cell. The resulting compounds may then be used to treat a chronic intraocular disorder.

A useful first step for identifying a compound for use in the treatment of a chronic intraocular disorder includes first identifying a compound that binds to Chlamydia pneumoniae or a Chlamydia pneumoniae specific agent. Various methods can be used, for example, the method can include immobilizing Chlamydia pneumoniae or a Chlamydia pneumoniae specific agent and incubating the immobilized Chlamydia pneumoniae or a Chlamydia pneumoniae specific agent with a test compound. Following the incubation period, unbound test compound is washed away and the bound test compound detected.

Once a Chlamydia pneumoniae binding compound has been identified, its ability to disrupt Chlamydia pneumoniae infectivity can be assayed. Such an assay can be performed by adding the compound to human monocyte-derived macrophages or RPE cells and then adding various titers of Chlamydia pneumoniae to the cells. The efficacy of the compound to inhibit Chlamydia pneumoniae infection of the cells can then be measured. Control reactions which do not contain the compound can be performed in parallel.

Pharmaceutical Formulations and Administration

The type and dosage of an anti-Chlamydia pneumoniae agent, such as an antibiotic, that should be administered to an individual depends upon various factors including, for example, the age, weight, gender, and health of the individual to be treated, as well as the type and/or severity of the particular disorder to be treated. The antibiotic preferably is administered in an amount and for a time sufficient to prevent infection by Chlamydia pneumoniae or kill, inactivate or otherwise prevent the progression of or survival of Chlamydia pneumoniae. The formulations, both for veterinary and for human medical use, typically include an active compound in association with a pharmaceutically acceptable carrier or excipient.

The carrier should be acceptable in the sense of being compatible with the other ingredients of the formulations and not deleterious to the recipient. Pharmaceutically acceptable carriers, in this regard, are intended to include any and all solvents, dispersion media, coatings, isotonic and absorption delaying agents, and the like, that are compatible with pharmaceutical administration. The use of such media and agents for pharmaceutically active substances is known in the art. Supplementary active compounds (identified or designed according to the invention and/or known in the art) also can be incorporated into the formulations. The formulations may conveniently be presented in dosage unit form and may be prepared by any of the methods well known in the art of pharmacy/microbiology. In general, some formulations are prepared by bringing the active molecule into association with a liquid carrier or a finely divided solid carrier or both, and then, if necessary, shaping the product into the desired formulation.

A pharmaceutical composition of the invention should be formulated to be compatible with its intended route of administration. Examples of routes of administration include local or systemic routes. Local routes include, for example, topical application to the eye, or intraorbital, periorbital, sub-tenons, intravitreal and transscleral delivery. Systemic routes include, for example, oral or parenteral routes, or alternatively via intramuscular, intravenous, intradermal, inhalation, transdermal (topical), transmucosal, and rectal routes.

Formulations suitable for oral or parenteral administration may be in the form of discrete units such as capsules, gelatin capsules, sachets, tablets, troches, or lozenges, each containing a predetermined amount of the antibiotic; a powder or granular composition; a solution or a suspension in an aqueous liquid or non-aqueous liquid; or an oil-in-water emulsion or a water-in-oil emulsion. Formulations suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). Sterile injectable solutions can be prepared by incorporating the active compound in the required amount in an appropriate solvent with one or a combination of ingredients enumerated above, as required, followed by filter sterilization. Formulations suitable for topical administration, including eye treatment, include liquid or semi-liquid preparations such as liniments, lotions, gels, applicants, oil-in-water or water-in-oil emulsions such as creams, ointments or pastes; or solutions or suspensions such as drops. Formulations for topical administration to the skin surface can be prepared by dispersing the drug with a dermatologically acceptable carrier such as a lotion, cream, ointment or soap. For inhalation treatments, inhalation of powder (self-propelling or spray formulations) dispensed with a spray can, a nebulizer, or an atomizer can be used. Such formulations can be in the form of a fine powder for pulmonary administration from a powder inhalation device or self-propelling powder-dispensing formulations.

In therapeutic use for treating, or combating Chlamydia pneumoniae infections in mammals, the active ingredients typically are administered orally, parenterally and/or topically at a dosage to obtain and maintain a concentration, i.e., an amount, or blood-level or tissue level of active ingredient which will be anti-microbially effective, i.e., kills, inactivates, or otherwise prevents the progression of a Chlamydia pneumoniae infection. Generally, an effective amount of dosage of active molecule will be in the range of from about 0.1 mg/kg to about 100 mg/kg, optionally from about 1.0 mg/kg to about 50 mg/kg of body weight/day. The amount administered likely will depend on such variables as the type and extent of disease or indication to be treated, the overall health status of the particular patient, the relative biological efficacy of the compound delivered, the formulation of the drug, the presence and types of excipients in the formulation, and the route of administration. Also, it is to be understood that the initial dosage administered may be increased beyond the above upper level in order to rapidly achieve the desired blood-level or tissue level, or the initial dosage may be smaller than the optimum and the daily dosage may be progressively increased during the course of treatment depending on the particular situation. If desired, the daily dose may also be divided into multiple doses for administration, for example, two to four times per day.

Exemplary anti-Chlamydial vaccines include, for example, Chlamydial DNA vaccines, live attenuated Chlamydia pneumoniae organisms, Chlamydial glycolipid exoantigen, and recombinant Chlamydial subunit vaccine. The vaccines may be administered using routes of administration and dosages known to those skilled in the art.

In light of the foregoing general discussion, the specific examples presented below are illustrative only and are not intended to limit the scope of the invention. Other generic and specific configurations will be apparent to those persons skilled in the art.

### III. EXAMPLES

### Example 1

### Serological Studies Showing a Correlation Between Chlamydia Pneumoniae Infection and the Onset of Age-related Macular Degeneration

Patients, all of whom were older than 55 years, were enrolled consecutively to either a case group of patients having age-related macular degeneration (ARMD patients) or a control group of patients without age-related macular degeneration (non-ARMD patients). The case group consisted of 25 patients with clinical evidence of age-related macular degeneration as determined by funduscopy. The control group consisted of 18 patients without clinical evidence of age-related macular degeneration by funduscopy.

Interview and chart-review data were gathered on several risk factors for age-related macular degeneration and cardiovascular disorders in order to assess potential confounding influences. Assessed factors included age, sex, as well as a history of smoking, diabetes, hypertension, hyperlipidemia, and coronary artery disease. Tobacco use was defined as current use or past smoking history of more than 5 pack-years. Diabetes was defined as a fasting blood sugar level greater than 126 mg/dL on two separate occasions, a glycosylated hemoglobin level greater than 7.5%, or use of antidiabetic therapy. Hypertension was defined as a history of systolic blood pressure higher than 160 mm Hg, diastolic blood pressure higher than 90 mm Hg, or use of antihypertensive therapy. Hyperlipidemia was defined as a history of total cholesterol greater than 200 mg/dL, a low-density lipoprotein level greater than 130 mg/dL, or use of lipid-lowering therapy. A history of coronary artery disease was noted if the patient had a history of stable angina, unstable angina, myocardial infarction, coronary angioplasty, or coronary artery bypass grafting.

Age-related macular degeneration was classified into non-neovascular or neovascular forms of the disease. Non-neovascular age-related macular degeneration was defined as macular drusen or the presence of geographic atrophy without choroidal neovascularization or scarring. Neovascular age-related macular degeneration was defined as the appearance of a choroidal neovascular membrane (CNVM) or scar on funduscopy and angiography.

Ten milliliters of blood were collected by venipuncture, and the serum was separated and stored frozen at −70° C. until use. The serum was tested for the presence of a variety of different antigens. Escherichia coli Hsp10 and Hsp60 (GroES and GroEL) were obtained from Stressgen Biotechnologies Corp. (Victoria, Canada). Chlamydia trachomatis Hsp10 and Hsp60 were purified by techniques known in the art (Yuan et al. (1992) INFECT. IMMUN. 60:2288-2296; La Verda et al. (1997) INFECT. IMMUN. 35:1209-1215). Chlamydia pneumoniae whole organisms (isolate TW183) were grown in HeLa cells, and the elementary bodies (EBs) harvested (Caldwell et al. (1981) INFECT. IMMUN. 31:1161-1176) and stored at −80° C. until use.

The antigens were detected via an enzyme-linked immunosorbent assay (ELISA) essentially as described (La Verda et al. (2000) INFECT. IMMUN. 68:303-309). Briefly, Immunolon 2 plates (Dynex Technologies, Chantilly, Va.) were coated with 0.5 μg of each antigen in PBS for 48 hours at 4° C. After this period, plates were washed 3 times with buffer containing PBS and 0.1% Tween 20, using a Labsystems Wellwash 4 Mk 2 plate washer (Labsystems Inc., Helsinki, Finland), then blocked for 90 minutes at 37° C. with PBS, 3% ovalbumin (grade II), and 0.1% Tween 20. The plates then were washed 3 times and incubated for 1 hour at 37° C. with a 1:250 dilution of patient sera in PBS, 0.1% ovalbumin (grade V), and 0.05% Tween 20. Following this step, the plates were washed 3 times, followed by incubation with alkaline phosphatase-conjugated goat anti-human immunoglobulin G (Jaclcson Immunoresearch Laboratories, West Grove, Pa.) for 30 minutes at 37° C. Finally, the plates were washed 3 times, followed by a rinse with Tris-buffered saline. The substrate P-nitrophenylphosphate (SigmaFAST tablets; Sigma Chemical Co., St Louis, Mo.) was added and incubated for 30 minutes at 37° C. Absorbance was read as optical density (OD) at 405 nm on a Perkin Elmer HTS 7000 Bio Assay Reader (Perkin Elmer Systems, San Francisco, Calif.). For each serum, the optical density (OD) value of a PBS-coated well that had no antigen (antigen-blank) was subtracted from the values for all test wells for that antigen. Triplicate-blanked test OD values for each antigen were averaged and reported for each patient. Laboratory personnel performing the ELISA test were blinded to clinical information on the patients.

The ELISA results of seroreactivity to each antigen were reported as OD from the assay measurements. Seroreactivity to each antigen between case and control groups was evaluated by 2-tailed t tests. Correlation between seroreactivity and age was assessed by a linear regression analysis. Multivariate regression was used to adjust for those variables independently associated with age-related macular degeneration. Sigmastat 2.0 (SPSS Inc., Chicago, Ill.) software was used to calculate P values from 2-tailed t tests and regression models. Sigmaplot 7.0 (SPSS) and Prism (GraphPad Software Inc., San Diego, Calif.) software were used to graph data.

Several risk factors for age-related macular degeneration and cardiovascular disease were evaluated. Univariate analysis confirmed that established risk factors for age-related macular degeneration occurred more frequently in patients as compared with controls (see Table 1; age: P<0.001; smoking: P=0.05). Frequency of hypertension, diabetes, coronary artery disease, and hyperlipidemia was similar between the 2 groups.

TABLE I

| | | Subjects | Subjects | |
|---|---|---|---|---|
| | | without | with | |
| | All Subjects | ARMD | ARMD | |
| | (n = 43) | (n = 18) | (n = 25) | P Value |
| Demographics | | | | |
| Age | 74.6 ± 8.5* | 68.7 ± 7.7 | 78.8 ± 6.2 | <0.001** |
| Male gender (%) | 98 | 100 | 96 | 0.403 |
| Potential Risk | | | | |
| Factors | | | | |
| Hypertension | 81 | 72 | 88 | 0.198 |
| Diabetes | 37 | 44 | 32 | 0.417 |
| Coronary Artery | 42 | 33 | 48 | 0.348 |
| Disease | | | | |
| Smoking | 51 | 33 | 64 | 0.049** |
| Hyperlipidemia | 70 | 66 | 72 | 0.715 |

*Plus - minus values are means ± standard deviation.
**P values less than 0.05 are presented in bold.

The seroreactivity to Chlamydia pneumioniae elementary bodies (EBs), Chlamydia trachomatis antigens (cHsp10, cHsp60), and Escherichia coli antigens (GroES, GroEL) is summarized in Table 2 below.

TABLE 2

| Antibody | | Subjects | | |
|---|---|---|---|---|
| Titers | | without | Subjects with | |
| (O.D. | All Subjects | ARMD | ARMD | P |
| reading) | (n = 43) | (n = 18) | (n = 25) | Value |
| Anti- | 0.069 ± 0.303* | 0.127 ± 0.451 | 0.026 ± 0.112 | 0.287 |
| cHsp10 | | | | |
| Anti- | 0.131 ± 0.307 | 0.083 ± 0.314 | 0.166 ± 0.303 | 0.391 |
| GroES | | | | |
| Anti- | 0.213 ± 0.338 | 0.241 ± 0.457 | 0.193 ± 0.226 | 0.651 |
| cHsp60 | | | | |
| Anti- | 0.338 ± 0.350 | 0.297 ± 0.349 | 0.368 ± 0.355 | 0.516 |
| GroEL | | | | |
| Anti-C. | 0.374 ± 0.274 | 0.276 ± 0.159 | 0.444 ± 0.319 | 0.047** |
| pneu- | | | | |
| moniae | | | | |

*Plus - minus values are means ± standard deviation.
**P values less than 0.05 are shown in bold.

O.D., optical density; cHsp10, Chlamydial heat shock protein 10; cHsp60, Chlamydial heat shock protein 60; GroES and GroEL, Escherichia coli molecular chaperones.

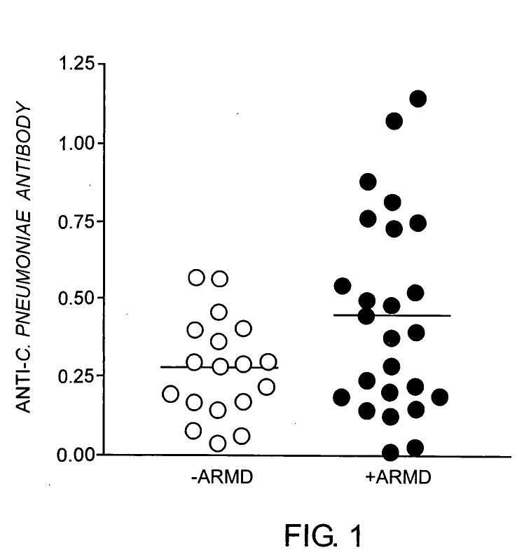

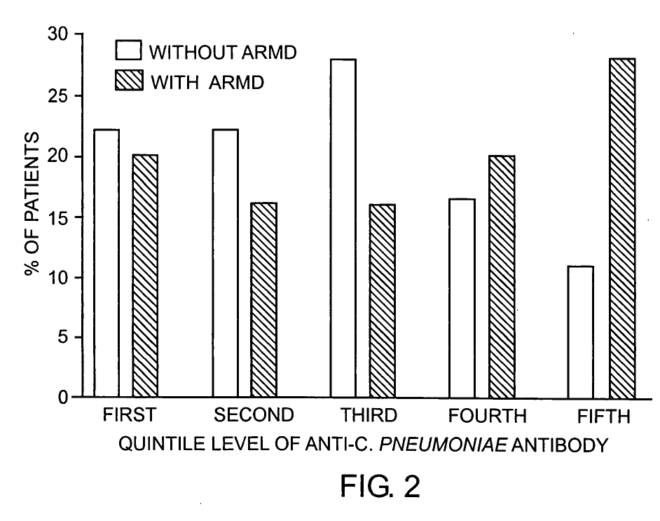

Significantly increased levels of antibodies to Chlamydia pneumoniae EBs were present in case patients compared with control patients (see, Table 2; P=0.05 and FIG. 1). In contrast, antibody titers to two Chlamydia trachomatis antigens (cHsp10 and cHsp60) and two Escherichia coli antigens (GroES and GroEL) were similar between case and control patients (see, Table 2). When anti-C pneumoniae antibodies were examined by quintiles, seven (28%) of twenty five patients with age-related macular degeneration and two (11%) of eighteen patients without age-related macular degeneration had levels measured in the highest 5th quintile (FIG. 2). Patients in the highest quintiles were similar in age (P=0.57) and history of smoking (P=0.25) as compared with patients in the lower quintiles.

Additional analyses were performed to determine if age and smoking may have confounded the association between antiChlamydia pneumoniae antibodies and age-related macular degeneration. Linear regression showed no correlation between age as a function of anti-Chlamydia pneumoniae antibodies for all patients (r²=0.002; P=0.80), patients with age-related macular degeneration (r²=0.083; P=0.16), or patients without age-related macular degeneration (r²<0.001; P=0.95). To determine if smoking was a confounder, antibody levels of both groups were analyzed by subgroups of smokers and nonsmokers. Nonsmokers with age-related macular degeneration were more likely to have higher anti-Chlamydia pneumoniae antibodies compared with nonsmokers without age-related macular degeneration (P=0.03). In addition, multivariate analysis adjusting for age and smoking showed that anti-Chlamydia pneumoniae antibodies remained significantly associated with age-related macular degeneration (P=0.05).

Based on these studies, a serlogical association was found between age-related macular degeneration and anti-Chlamydia pneumoniae antibodies (P=0.05), but not between age-related macular degeneration and the anti-Chlamydia trachomatis or anti-E. coli heat shock protein antibodies. The association remained statistically significant after adjusting for age and smoking, both established risk factors for age-related macular degeneration.

These experiments show that Chlamydia pneumoniae infection is associated with the onset of age-related macular degeneration.

### Example 2

### Tissue Studies Showing a Correlation Between Chlamydia Pneumoniae Infection and the Onset of Age-Related Macular Degeneration

To further substantiate the presence of Chlamydia pneumoniae in the CNVM of patients diagnosed with age-related macular degeneration, serial sections of CNVM specimens were isolated from nine patients and analyzed either by PCR to detect the presence of Chlamydia pneumonia specific nucleic acids in the samples or by immunohistochemistry using an anti-Chlamydia pneumoniae monoclonal antibody to detect the presence of a Chlamydia pneumonia specific antigen in the samples.

Materials and Methods

Materials. Unless otherwise noted, all reagents were purchased from Sigma (St. Louis, Mo.).

Tissue Preparation. Human studies committee approval was obtained at the Massachusetts Eye & Ear Infirmary and Massachusetts General Hospital (Boston, Mass.). Sterile technique was used to handle all specimens. Surgical excision of submacular CNVM was performed in nine eyes of nine patients according to previously described techniques (Lambert et al. (1993) CURR. OPIN. OPHTH. 4: 19-24). Immediately after surgical excision, CNVM were placed in 4% paraformaldehyde overnight, washed in PBS at 4° C. for 4 hours, dehydrated with a series of graded alcohol solutions, and embedded in paraffin. In addition, seven eyes without age-related macular degeneration enucleated for uveal melanoma were formalin-fixed, paraffin embedded and axially sectioned at the optic nerve for immunohistochmeistry. Furthermore, nine frozen whole eyes from patients without age-related macular degeneration (ages 70-85) were obtained from the New England Eye Bank (Boston, Mass.). Eyes were thawed, sectioned at the coronal equator, and examined under a dissecting microscope to ensure absence of drusen and CNVM. Retina, choroid and iris were meticulously dissected under sterile conditions and placed in separate tubes for DNA extraction.

For PCR of age-related macular degeneration CNVM, 20-30 pim sections were obtained from each specimen and total DNA was extracted with the Qiamp DNA mini kit (Qiagen, Valencia, Calif.), using a modification of the manufacturer's protocol to remove paraffin (Wu et al. (2002) APPL. IMMUNHIST. MOL. MORPHOL. 10:269-274). For PCR of control Eye Bank retina, choroid and iris specimens, DNA was extracted from approximately 1-2 mg of each specimen using the Qiamp DNA mini kcit. For immunohistochemistry, paraffin-embedded CNVM specimens were serially sectioned at 4-5 μm thickness, placed on glass slides and deparaffinized by xylene—alcohol treatment. Enough tissue was available to obtain two to six sections per specimen. When available, two to three adjacent sections were placed on each slide to be stained simultaneously.

PCR and Automated Sequencing. Meticulous care was taken to prevent contamination and amplicon carryover. All PCR was done under sterile conditions. Positive control DNA was dispensed only after all other specimen tubes were capped and removed from the culture hood. All PCR tubes, Qiamp DNA extraction kit reagents and spin columns were treated with UV light (Strategene UV crosslinker, Strategene, La Jolla, Calif.) to ensure absence of amplifiable DNA; PCR did not amplify any product from UV-treated tubes and reagents when targeting a universal bacterial 16S ribosomal RNA sequence (Relman et al. (1992) N. ENG. J. MED. 327: 293-301).

DNA was amplified with touchdown-nested PCR with primers, CP1-CP2/CPC-CPD, targeting the Chlamydia pneumoniae major outer membrane protein sequence. The primers were as follows: CP1-TTA CAA GCC TTG CCT GTA GG [SEQ. ID No.: 1]; CP2-GCG ATC CCA AAT GTT TAA GGC [SEQ. ID No.: 2]; CPC-TTA TTA ATTIGAT GGT ACA ATA [SEQ. ID No.: 3]; CPD-ATC TAC GGC AGT AGT ATA GTT [SEQ. ID No.: 4] as described in Tonget al. (1993) J. CLIN. PATHOL. 46:313-317. For PCR using CP1-CP2 with nested primer pair CPC-CPD, the conditions were as follows: the first round of amplification employed 1.5 mM MgCl₂, 0.4 RM primers, and 0.625 U of Taq polymerase and involved 20 cycles of 1 minute at 94° C., 1 minute at 64° C. minus 0.5° C. per cycle, and 1 minute at 72° C. plus an additional 20 cycles of 1 minute at 94° C., 1 minute at 55° C., and 1 minute at 72° C. The PCR products amplified by the outer primers (CP1-CP2) were diluted 1:10, and a volume of 2.5 μl was added to a new 25-μl PCR mixture for a second amplification with nested primer pair CPC—CPD. The second round of amplification employed 3 mM MgCL₂, 1 μM primers, and 0.625 U of Taq polymerase and involved 30 cycles of 1 minute at 94° C., 1 minute at 50° C. and 1 minute at 72° C. 0.1-1% bovine serum albumin was used in each reaction to allow amplification in the presence of melanin, a PCR inhibitor present in uveal tissue (Eckhart et al. (2000) BIOCHIM. BIOPHYS. RES. COMM. 271:726-730). Samples were electrophoresed on 1.5% agarose gels, stained with ethidium bromide, and visualized under UV light. Ethidium bromide—stained gel bands were cut with a sterile razor blade, and the band DNA was extracted and purified with a Qiagen gel extraction kit (Valencia, Calif.). Purified DNA was analyzed by an automated sequencer at the DNA Sequencing Center for Vision Research (DSCVR) (Massachusetts Eye and Ear Infirmary, Boston, Mass.). Sequencing was performed first with the CPD primer, and sequence identity was confirmed by re-sequencing with the CPC primer. Sequences were analyzed with Chromas software (Technelysium Pty Ltd, Helensvale, Australia) and compared with sequences in the National Center for Biotechnology Information database using Basic Local Alignment Search Tool (BLAST). Data are representative of at least two separate experiments.

Immunohistochemistry. Antigen retrieval was performed by treatment with target retrieval solution (Dalco S 1700; Carpinteria, Calif.) in a water bath at 95° C. for 20 minutes. Sections were incubated for one hour at room temperature with RR-402 mouse monoclonal anti-C pneumoniae antibody (1:50 dilution, Dakocytomation, Carpenteria, Calif.). Negative controls either omitted the primary antibody or substituted an isotype—specific monoclonal antibody at a similar concentration. Positive controls consisted of human RPE cells or human monocyte-derived macrophages infected with Chlamydia pneumoniae and fixed in formalin. A standard immunohistochemistry protocol then was performed with the HRP-AEC anti-mouse cell and tissue staining kit (Dako AP EnVision System, Dakcytomation) according to the manufacturer's recommendations. All incubations were carried out in a humidifier chamber. After incubation, slides were washed in PBS or tris buffered saline (TBS), counterstained with Mayer's hematoxylin (Sigma), mounted with aqueous mounting medium (Farmount, Dakocytomation) and examined by light microscopy.

Results

Detection of Chlamydia Pneumoniae in CNVM by PCR

Nine CNVM surgically removed from patients with age-related macular degeneration and nine eyes without evidence of age-related macular degeneration were examined by touchdown-nested PCR (Tong (1993) supra) for the presence of Chlamydiae pneumoniae DNA. Meticulous care was taken to prevent contamination and amplicon carryover, including use of sterile technique, UV-irradiation of all DNA extraction reagents and tubes, and use of low Chlamydia pneumoniae DNA levels for positive controls.

Chlamydia pneumoniae DNA was not detected from any tissue isolated from the nine control eyes. In contrast, DNA extracted from two of nine CNVM amplified the expected Chlamydia pneumoniae major outer membrane gene segment. A negative control (Chlamydia trachomatis serovar A DNA) and postive control (Chlamydia pneumoniae AR-39 DNA) gave expected results in each reaction. In addition, the original primer pair produced products of the expected size for each positive reaction, indicating that the final products did not result from amplicon carryover during nested PCR. For additional confirmation of the specificity of the PCR, the amplified product bands were extracted from the gel, purified and subjected to automated sequencing, revealing 100% identity to the expected Chlamydia pneumoniae major outer membrane gene segment. These data indicate that Chlamydia pneumoniae DNA is present in some Chlamydial neovasculature secondary to age-related macular degeneration, and suggest that Chlamydia pneumoniae infection may be associated with the development of age-related macular degeneration.

Detection of Chlamydia Pneumoniae in CNVM by Immunohistochemistry

To further substantiate the presence of Chlamydia pneumoniae in the CNVM of patients diagnosed with age-related macular degeneration, serial sections of CNVM specimens isolated from the nine patients were stained with an anti-Chlamydia pneumoniae monoclonal antibody (RR402) via immunohistochemistry.

Results showed that four of the nine specimens showed evidence of Chlamydia pneumoniae. A specimen was considered positive only if intracellular staining was observed in corresponding locations of two adjacent serial sections. Staining was not noted in CNVM specimens incubated with an isotype-specific control antibody or without primary antibody. In addition, an internal limiting membrane (ILM) peel section that was incubated with anti-Chlamydia pneumoniae antibody as a negative control did not show evidence of staining. Furthermore, no staining was observed in serial sections of five CNVM surgically extracted from patients without evidence of age-related macular degeneration, or from axial sections of seven non-age-related macular degeneration eyes enucleated for uveal melanoma. Some staining in age-related macular degeneration CNVM specimens localized within lipofuscin-laden cells characteristic of RPE. Two of the four positive specimens were extracted from patients previously treated with photodynamic therapy. Because CNVM resulting from non-age-related macular degeneration disease tend to occur in younger patients than age-related macular degeneration CNVM, immunohistochemistry control specimens were no age-matched to age-related macular degeneration CNVM.

In summary, Chlamydia pneumoniae was detected in two out of nine samples of CNVM secondary to age-related macular degeneration samples via PCR and four out of nine samples of CNVM secondary to age-related macular degeneration via immunohistochemistry. In contrast, none of the samples from patients without age-related macular degeneration showed evidence for Chlamydia pneumoniae. The data support the theory that Chlamydia pneumoniae is a risk factor for the development of age-related macular degeneration.

### Example 3

### Chlamydia pneumoniae Infects Certain Cells, and Induces Production of Angiogenic Immunomodulators

This experiment was performed to determine if Chlamydia pneumoniae can alter cell function in ways that cause age-related macular degeneration. To perform this experiment, human monocyte-derived macrophages and RPE cells were infected with varying doses of Chlamydia pneumoniae and assayed for the production of pro-angiogenic and inflammatory immunomodulators.

Human peripheral blood mononuclear cells were isolated by Ficoll-Hypaque centrifugation from healthy donor blood. Monocytes were separated from lymphocytes by incubating cells at 37° C. for 1 hour in RPMI-1640 medium (Sigma) followed by washing 5× with HBSS (Sigma). In separate experiments, isolated cells were >90% monocytes by anti-CD14 staining (Kalayoglu et al. (1998) J. INFECT. DIS. 177:725-729) and viability was >95% as assessed by trypan-blue dye exclusion. Monocytes were cultured at 37° C. for 5-7 days in RPMI-1640 medium supplemented with 10% FBS, 10 ug/ml gentamycin and 25 mM HEPES buffer to allow maturation into macrophages. Human RPE cells (ARPE-19 cell line, ATCC, Manassas, Va.) were propagated in 1:1 DMEM/Ham's F12 media (ATCC) supplemented with 10% FBS and 10 ug/ml gentamycin. All cells were plated in 96-well microtiter wells or LabTek slides for the experiments (Nalge Nunc International, Rochester, N.Y.). Cells were mock-infected or infected with varying doses of Chlamydia pneumoniae by incubating for 2 hours at 37° C. in 50 μl medium, then cultured for 10 to 64 hours in 200 μl medium. Media were assayed by commercially-available ELISA kits for the presence of VEGF (R&D Systems), IL-8 and MCP-1 (Anogen, Mississauga, Ontario). Separate cell protein assays indicated that infection did not globally increase protein secretion by the tested cell types. Cells were fixed in methanol and stained with the Pathfinder FITC-conjugated anti-Chlamydial monoclonal antibody (Bio-Rad Laboratories, Hercules, Calif.). Assays were conducted in triplicate and data are representative of at least two similar experiments.

Chlamydiae pneumoniae was found to productively infect human macrophages and RPE cells. The organism established large, multiple inclusions within human RPE cells. In contrast, although monocyte-derived macrophages infected with C. pneumoniae at similar doses supported growth of the pathogen, well-defined inclusions were not seen in this cell type.

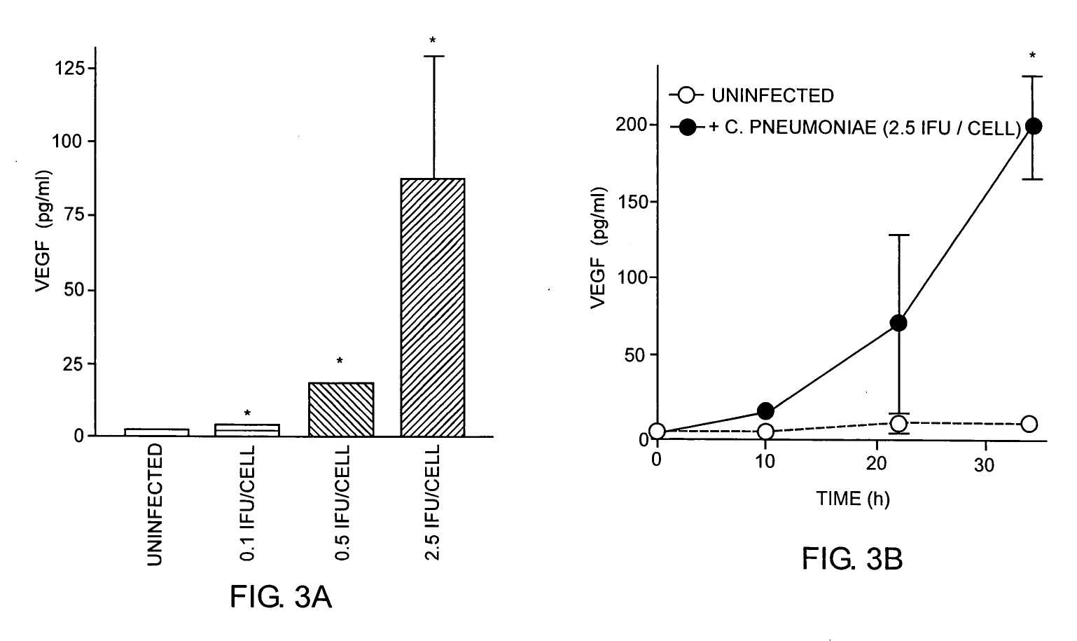

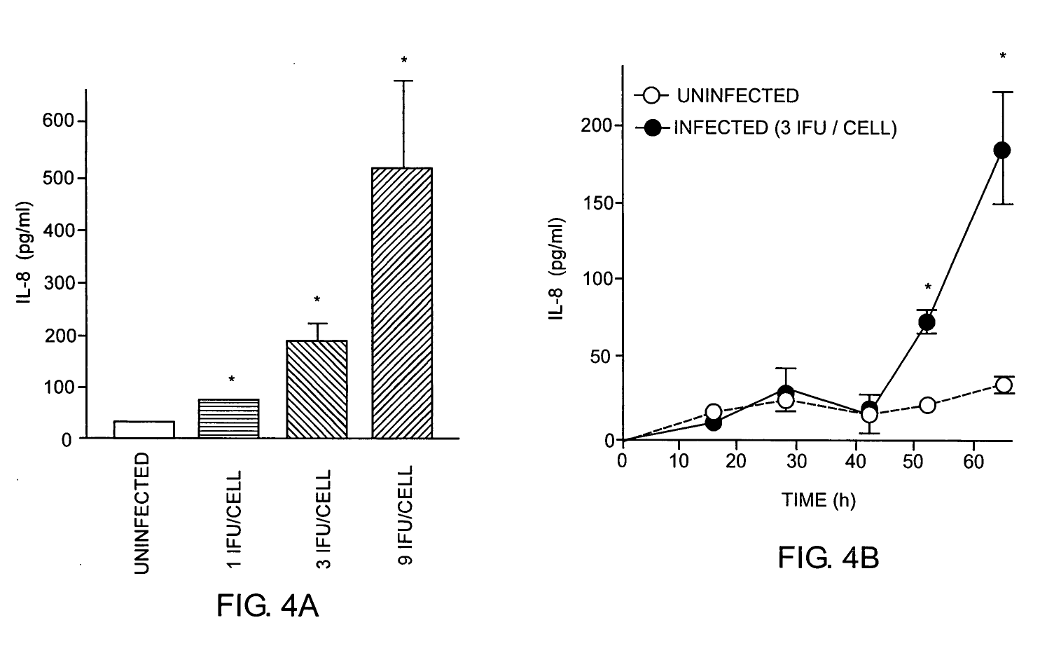

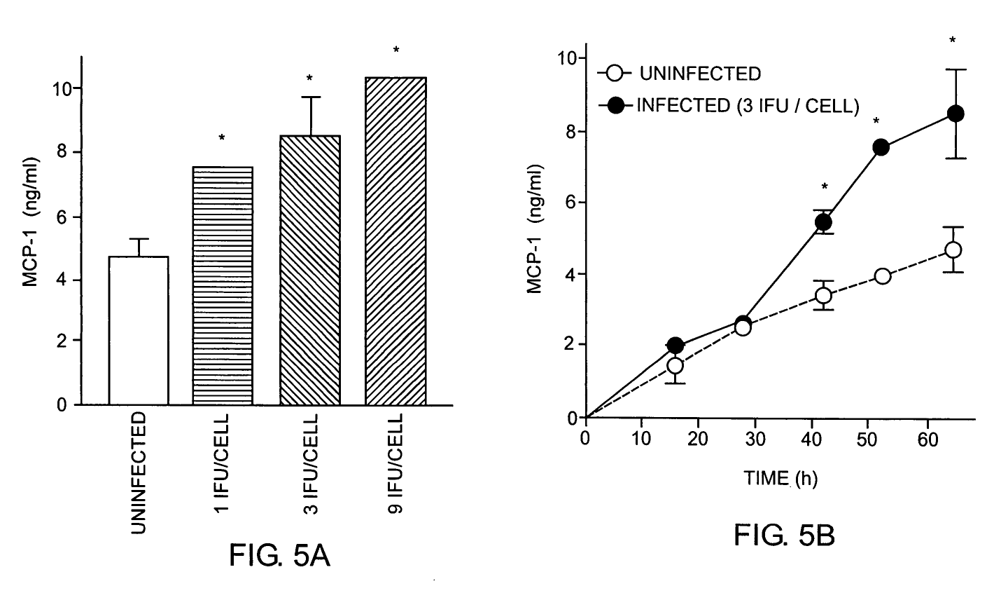

Infected cells also were assayed for increased production of angiogenic immunomodulators. Chlamydia pneumoniae dramatically induced VEGF by macrophages in a dose (FIG. 3a) and time (FIG. 3b) dependent manner. Higher doses of C. pneumoniae led to an approximately 50-fold increase in VEGF secretion, and stimulation was evident within 24 hours of infection. In contrast, human RPE cells (ARPE-19 cell line) constitutively expressed VEGF, and Chlamydiae pneumoniae did not appreciably increase VEGF production by this cell type. However, Chlamydiae pneumoniae—mediated dysregulation of RPE function was evident by a dose and time-dependent increase in IL-8 (FIGS. 4a and 4b, respectively) and MCP-1 (FIGS. 5a and 5b, respectively) by infected ARPE-19 cells. Higher doses of Chlamydia pneumoniae led to a 10 to 20-fold increase in IL-8 levels and a 2-fold increase in MCP-1 production.

In summary, Chlamydia pneumoniae was found to induce key angiogenic cytokines by ARPE-19 cells and macrophages in a dose and time dependent manner. One of these cytokines, VEGF, has been localized to surgically extracted CNVM from patients with age-related macular degeneration. These data are consistent with the theory that Chlamydia pneumoniae infection contributes to the pathogenesis of age-related macular degeneration.

### Example 4

### Association Between Chlamydial Heat Shock Protein 60 (cHsp60) and Age-Related Macular Degeneration

Sera from 70 individuals without age-related macular degeneration, 43 patients with the dry form of age-related macular degeneration, and 51 patients with the neovascular form of age-related macular degeneration were collected and assayed for the presence of IgG antibody to cHsp60. Potential confounding variables such as gender, coronary artery disease, diabetes mellitus, smoking, hypertension and hypercholesterolemia were evaluated by age-adjusted bivariate logistic regression models. An association between age-related macular degeneration and Chlamydial infection was evaluated by age-adjusted multivariate logistic regression comparing control patients without age-related macular degeneration to patients with (i) the dry form of age-related macular degeneration, (ii) the neovascular form of age-related macular degeneration, or (iii) both the dry and neovascular forms of age-related macular degeneration combined.

Preliminary results suggest that the concentration of cHsp60 IgG in the samples correlates with the presence of any type of age-related macular degeneration (OR: 2.8, 95% CI: 0.97-8.36; P=0.058). Also, the preliminary results suggest that the concentration of cHsp60 in the samples correlates more strongly with the dry form of age-related macular degeneration (OR: 3.7, 95% CI: 1.003-13.99; P=0.049) than with the neovascular form of age-related macular degeneration (OR: 2.1, 95% CI: 0.64-6.94; P=0.22).

### INCORPORATION BY REFERENCE

The entire disclosure of each of the publications and patent documents referred to herein is incorporated by reference in its entirety for all purposes to the same extent as if each individual publication or patent document were so individually denoted.

### Equivalents

The invention may be embodied in other specific forms without departing form the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein

What is claimed is:

1. A method of determining whether a mammal is at risk of developing, or has, a chronic intraocular disorder, the method comprising:
detecting in a sample from the mammal the presence of Chlamydia pneumoniae or a Chlamydia pneumoniae specific agent, the presence of which is indicative that the mammal is at risk of developing, or has, the chronic intraocular disorder.

2. The method of claim 1, wherein the sample is a tissue or body fluid sample.

3. The method of claim 2, wherein the body fluid sample is selected from the group consisting of blood, serum and plasma.

4. The method of claim 2, wherein the tissue is choroid or retina.

5. The method of claim 1, wherein the Chlamydia pneumoniae specific agent is a Chlamydia pneumonia specific nucleic acid or protein.

6. The method of claim 5, wherein the Chlamydia pneumoniae specific agent is a host antibody.

7. The method of claim 6, wherein the antibody is an anti-Chlamydia pneumoniae elementary body antibody.

8. The method of claim 5, wherein the nucleic acid is detected by nucleic acid hybridization.

9. The method of claim 8, wherein the nucleic acid is amplified prior to detection.

10. The method of claim 1, wherein the disorder is age-related macular degeneration.

11. The method of claim 1, wherein the mammal is a human.

12. A method of determining whether a human is at risk of developing, or has, age-related macular degeneration, the method comprising:
detecting in a sample from the human the presence of Chlamydia pneumoniae or a Chlamydia pneumoniae specific agent, the presence of which is indicative that the human is at risk of developing, or has, age-related macular degeneration.

13. The method of claim 12, wherein the sample is a tissue or body fluid sample.

14. The method of claim 12, wherein the body fluid sample is selected from the group consisting of blood, serum and plasma.

15. The method of claim 13, wherein the tissue is choroid or retina.

16. The method of claim 12, wherein the Chlamydia pneumoniae specific agent is a host antibody.

17. The method of claim 16, wherein the antibody is an anti-Chlamydia pneumoniae elementary body antibody.

18. The method of claim 12, wherein the Chlamydia pneumoniae specific acid is a Chlamydia pneumoniae specific nucleic acid.

19. A method of preventing, slowing or stopping the progression of a chronic intraocular disorder, the method comprising the step of:
administering to a mammal suspected of having or developing a chronic intraocular disorder an effective amount of an anti-Chlamydia pneumoniae agent thereby to prevent, slow or stop the progression of the disorder.

20. The method of claim 19, wherein the disorder is age-related macular degeneration.

21. The method of claim 20, wherein the mammal is a human.

22. The method of claim 19, wherein the agent is an antibiotic.

23. The method of claim 19, wherein the agent is administered parenterally or 2 systematically.

24. The method of claim 19, wherein the agent is administered locally to an eye of the mammal.

25. The method of claim 19, comprising administering a plurality of anti-Chlamydia pneumoniae agents.

26. The method claim 25, wherein the plurality of agents are administered simultaneously or sequentially.

27. A kit comprising an agent for detecting Chlamydia pneumoniae or a Chlamydia pneumoniae specific agent and instructions on how to detect Chlamydia pneumoniae or a Chlamydia pneumoniae specific agent in a sample to determine if a mammal is at risk of developing, or has, a chronic intraocular disorder.

28. The kit of claim 27, wherein the chronic intraocular disorder is age-related macular degeneration.
